(19) United States
(12) Patent Application Publication
(10) Pub. No.: US 2025/0161675 A1
(43) Pub. Date: May 22, 2025

(54) TEMPORARY NERVE BLOCK DEVICE FOR ANIMAL DISTAL EXTREMITY

(71) Applicant: T-EQUALS-0 INNOVATION LAB LLC, Vista, CA (US)

(72) Inventors: Richard Wayne Clark, Norco, CA (US); Reghu Rajan Kunnath, Whittier, CA (US); Gary R. Cantu, Vista, CA (US); Ted P. Vlahos, Cody, WY (US)

(21) Appl. No.: 18/955,755

(22) Filed: Nov. 21, 2024

Related U.S. Application Data
(60) Provisional application No. 63/601,635, filed on Nov. 21, 2023.

(51) Int. Cl. A61N 1/36; A61N 1/04; A61N 2/00; A61N 2/02

(52) U.S. Cl. CPC A61N 1/36021; A61N 1/0408; A61N 1/0476; A61N 1/36031; A61N 2/002; A61N 2/008; A61N 2/02

(57) ABSTRACT

A temporary wearable device, in the form of a cuff with electronics and arrays of skin electrodes, attachable to a limb at specific locations above various joints. When activated, such as by wireless actuation, the device stimulates specific nerves of interest to temporarily block pain signals with quick response and in a reversible manner without any permanent nerve damage to the animal.

### CROSS-REFERENCE TO RELATED APPLICATIONS

This application relates to and claims the benefit of U.S. Provisional Application No. 63/601,635 filed Nov. 21, 2023 and entitled “TEMPORARY NERVE BLOCK DEVICE FOR ANIMAL DISTAL EXTREMITY”, the entire disclosure of which is wholly incorporated by reference herein.

### STATEMENT RE: FEDERALLY SPONSORED RESEARCH/DEVELOPMENT

Not Applicable

### BACKGROUND

### 1. Technical Field

The present disclosure relates generally to veterinary treatment devices, and more particularly, to a temporary nerve blocking device for distal extremities of animals.

### 2. Related Art

Joint injuries on animal limbs, such as equine limbs, are extremely common, caused by various factors such as trauma, inflammation, and/or infection. This causes pain to the horse and leads to lameness or limping, and several other short and long-term injuries.

The current standard practice for the diagnosis of the specific location of the internal injury such as that of a limb of the horse, begins with a veterinary professional identifying potentially impacted nerves and injecting a numbing agent to locally desensitize the nerve and temporarily block the pain sensation. The veterinary professional must then wait for the desensitization to complete, then directing the horse to repeat the physical movement while observing for changes in the gait. If the numbing agent was introduced at the correct, injured joint, the horse will not perceive the pain and a noticeable change in gait may be observed.

If the numbing agent was injected into the wrong joint, however, it is necessary to further wait for the numbing agent to fade. Then, the veterinary professional makes the next best guess as to the possibly injured joint, identifies the nerves above such joint, then administers the numbing agent again via injection. The physical movement test is then repeated once the numbing agent has completed the desensitization of the likely affected nerve.

As can be appreciated, this is an extremely slow and time-intensive process involving potentially repeated injections of a drug. This is often painful to the horse, leading to associated increased medical treatment costs. Furthermore, injecting numbing agents into the nerves can cause short-term to medium-term pain, as well as mild numbness for a potentially extended duration. Furthermore, multiple injections can also increase the risk of infection.

Accordingly, there is a need in the art for an improved diagnosis modality for identifying joint injuries, particularly in horse limbs. There is a need in the art for a non-invasive wearable device that can stimulate specific nerves of interest to block signals with a quick response while being readily reversible without risking permanent nerve damage.

### BRIEF SUMMARY

An electronic device that utilizes external neurostimulation using non-invasive skin electrodes to desensitize superficial nerves to block the transmission of pain signals to the brain for a temporary duration is disclosed. External high frequency signals may desensitize nerves, though identifying the exact nerve bundle, delivering the right type of excitation signal (pulse waveform, frequency, amplitude, duration, envelop parameters and the pattern) is specific and tailored for every application and joint, based on the type of nerve group and the part of the limb. The device is contemplated to be lightweight such that gait asymmetry does not result during use, as it remains on the limb during the nerve blocking operation as well as during evaluation for potential injuries. The optimum location on the skin to stimulate nerves is based upon an automatic identification of peak superficial neuroactivity with sensors that are spaced and spatially arranged electrodes. The signals delivered through the electrodes simulate the nerve bundle to temporarily desensitize for pain signals only without affecting motor signaling to the muscles.

According to one embodiment of the present disclosure, there may be a temporary nerve blocking device attachable to a limb of an animal. The device may include a strap for wrapping around the limb. The strap may be defined by a limb contact side and an external side. The device may also include an array of electrodes fixed to the strap. The electrical contacts thereof are exposed on the limb contact side of the strap, and are spaced apart along the strap and arranged in a predetermined pattern. There may also be a sensor monitor that is coupled to the strap and connected to one or more of the electrodes. Skin conductivity may be measured by the sensor monitor. The device may also include a signal generator that is coupled to the strap. The signal generator may have a plurality of electrode outputs connected to a respective one of the electrodes of the array. The signal generator may output a nerve blocking signal to one or more of the electrodes. Parameters of the nerve blocking signal may be a function of one or more limb parameters.

Another embodiment of the present disclosure may be a temporary nerve blocking device. There may be a strap for wrapping around a limb, as well as a plurality of electrodes fixed to the strap with electrical contacts thereof being exposed. There may also be a digital signal processor integrated circuit with one or more inputs and one or more outputs. The digital signal processor may be programmable to generate to the one or more outputs multiple variants of a nerve blocking signal. Each variant of the nerve blocking signal may be defined by one or more of the parameters of waveform type, frequency, amplitude, and phase, and duration. The device may further include a memory for storing parameter data for the multiple variants of the nerve blocking signal. There may also be a sensor monitor that is connected to the electrodes and to one of the one or more inputs. The device may include driver circuits that are connected to the outputs of the digital signal processor and amplify the nerve blocking signal for transmission to the electrodes.

The present disclosure will be best understood accompanying by reference to the following detailed description when read in conjunction with the drawings.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of an exemplary first embodiment of a temporary nerve blocking device;

FIG. 2 is a detailed view of a control module incorporated into various embodiments of the temporary nerve blocking device;

FIG. 3 is a side view of the first embodiment of the temporary nerve blocking device;

FIG. 4 illustrates additional details of the array of electrodes in an exemplary embodiment of the temporary nerve blocking device;

FIG. 5 is a perspective view of a second embodiment of the temporary nerve blocking device;

FIG. 6A and FIG. 6B illustrate possible locations along a limb of a horse where the temporary nerve blocking device may be placed;

FIG. 7 illustrates the placement of the temporary nerve blocking device on the limb of the horse;

FIG. 8 is a cross-sectional view of the limb of the horse with the temporary nerve blocking device attached thereto;

FIG. 9 is a partial perspective view of the limb of the horse with the temporary nerve blocking device attached thereto; and

FIG. 10 is a block diagram illustrating the components of the temporary nerve blocking device.

### DETAILED DESCRIPTION

The detailed description set forth below in connection with the appended drawings is intended as a description of the several presently contemplated embodiments of a temporary nerve blocking device and is not intended to represent the only form in which such embodiments may be developed or utilized. The description sets forth the functions and features in connection with the illustrated embodiments. It is to be understood, however, that the same or equivalent functions may be accomplished by different embodiments that are also intended to be encompassed within the scope of the present disclosure. It is further understood that the use of relational terms such as first and second and the like are used solely to distinguish one from another entity without necessarily requiring or implying any actual such relationship or order between such entities.

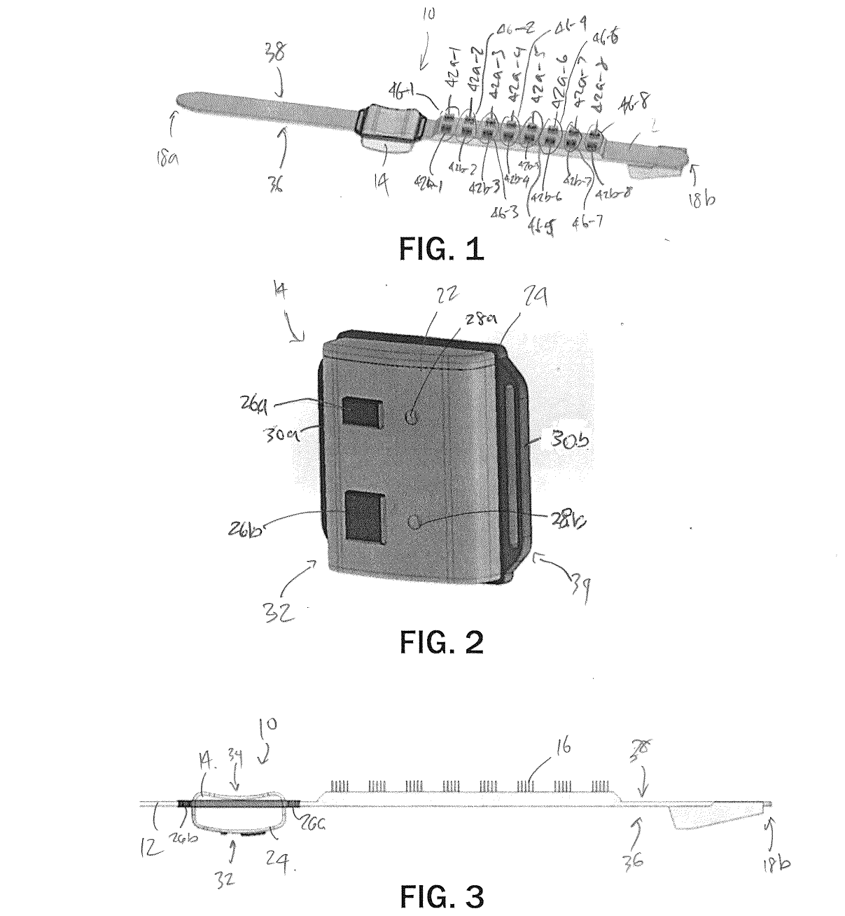

FIG. 1 depicts one embodiment of a temporary nerve blocking device 10, which includes a strap 12, a controller 14, and an array of electrodes 16 embedded in the strap 12. In the exemplary embodiment, the strap 12 is a single band of elastic fabric or other like material defined by a first end 18a and an opposed second end 18b. The second end 18b may incorporate a clasp 20 that receives the first end 18a of the strap 12 after being looped around the limb and can be locked at arbitrary positions. The specific length or position at which the clasp 20 is locked relative to the first end 18a of the strap 12 may define the overall working diameter thereof. The portion of the strap 12 proximal to the second end 18b includes the electrodes 16, and adjacent thereto is the controller 14.

As additionally shown in FIGS. 2 and 3, the controller 14 is defined by a housing 22 mounted to a frame 24. The housing 22 is understood to incorporate various electronic components that will be described in further detail below. Externally accessible on the housing 22 may be one or more buttons 26a, 26b for providing a limited set of inputs to the controller 14, as well as one or more indicators 28a, 28b that display limited outputs from the controller 14. The indicators 28 may be a simple light emitting diode that may be activated at different intensities and/or timing to indicate different information. The controller 14 may incorporate other output modalities such as display screens capable of showing alphanumeric characters, graphics, and so on. The frame 24 is defined by a pair of opposed strap loops 30a, 30b, through which the strap 12 may be passed to attach the housing 22 thereto.

According to one embodiment, the temporary nerve blocking device 10 is a standalone unit that can be attached to the limb of an animal such as a horse without being physically tethered to any other device for power or control purposes. Thus, the housing 22 may also incorporate a power source such as a battery, as well as a wireless communications modality that may establish a data to receive control instructions, parameter data, and so on. Additional details of the internal components of the controller 14 will be provided below.

The housing 22 may be defined by a front face 32 on which the buttons 26 and the indicators 28 are provided. The housing 22 also defines a rear face 34 that has a curved structure to accommodate a correspondingly curved structure of the limb. Along these lines, the strap 12 is likewise defined by an external face or side 36, and an internal or limb contact side 38, with the electrodes 16 being positioned on the limb contact side 38.

Although a specific embodiment of an adjustable strap 12 has been presented, this is by way of example only and not of limitation. Any other suitable adjustment modality may be substituted without departing from the scope of the present disclosure. Along these lines, while the various embodiments of the temporary nerve blocking device 10 are described in the context of treating a horse, with the sizing and other physical configuration being specific to the treatment of horses, this is also by way of example only. The temporary nerve blocking device 10 may be adapted to other animals, such as dogs, cats, and farm animals like. In this regard, it is deemed to be within the purview of those having ordinary skill in the art to make suitable modifications to the temporary nerve blocking device 10 to accommodate such other types of animals. Beyond animals, it is expressly contemplated that the temporary nerve blocking device may be adapted for use with human beings as well, particularly those who are unable to verbally communicate on anatomical features.

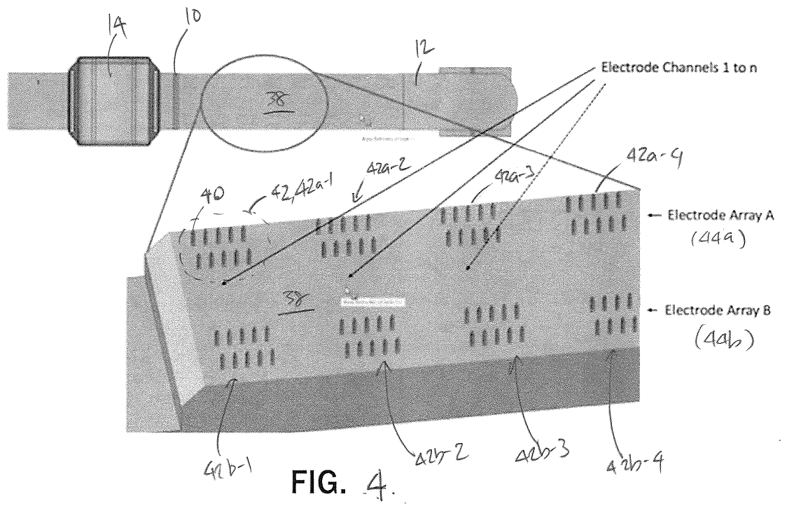

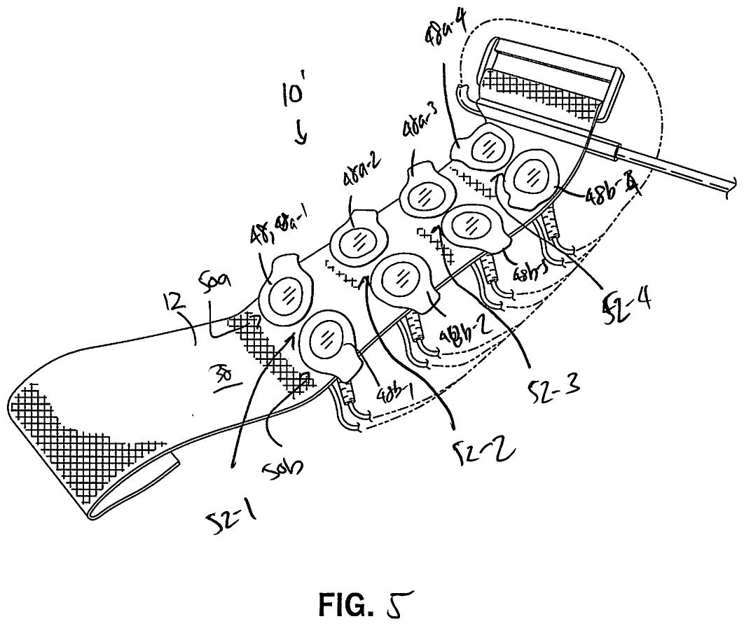

With additional reference to FIG. 4, in the first embodiment of the temporary nerve blocking device 10, the electrodes 16 are pin electrodes, with individual pin contacts 40 being exposed on the limb contact side. A group of individual pin contacts 40 are aggregated as a single electrode element 42, with each of the pin contacts 40 thereof being connected to a single input port or output port.

In turn, an arrangement of multiple electrode elements 42 along one side of the longitudinal axis of the strap 12 may be referenced as an array or array segment 44. FIG. 1 and FIG. 4 illustrate a first array segment 44a that includes a first electrode element 42a-1, a second electrode element 42a-2, a third electrode element 42a-3, a fourth electrode element 42a-4, a fifth electrode element 42a-5, sixth electrode element 42a-6, a seventh electrode element 42a-7, and an eighth electrode element 42a-8. The temporary nerve blocking device 10 also includes a second array or array segment 44b with a first electrode element 42b-1, a second electrode element 42b-2, a third electrode element 42b-3, a fourth electrode element 42b-4, a fifth electrode element 42b-5, sixth electrode element 42b-6, a seventh electrode element 42b-7, and an eighth electrode element 42b-8. Each of the eight electrode elements 42 in a given array segment 44 are spaced apart from each other by a predetermined distance.

Laterally adjacent electrode elements 42 in the first array segment 44a and the second array segment 44b may be referred to as electrode channels. Thus, the first electrode element 42a-1 and the first electrode element 42b-1 define a first electrode channel 46-1, the second electrode element 42a-2 and the second electrode element 42b-2 define a second electrode channel 46-2, the third electrode element 42a-3 and the second electrode element 42b-3 define a third electrode channel 46-3, the fourth electrode element 42a-4 and the fourth electrode element 42b-4 define a fourth electrode channel 46-4, the fifth electrode element 42a-5 and the fifth electrode element 42b-5 define a fifth electrode channel 46-5, the sixth electrode element 42a-6 and the sixth electrode element 42b-6 define a sixth electrode channel 46-6, the seventh electrode element 42a-7 and the seventh electrode element 42b-7 define a seventh electrode channel 46-7, and the eighth electrode element 42a-8 and the eighth electrode element 42b-8 define a sixth electrode channel 46-8. The individual electrode elements 42a of the first array segment 44a and the electrode elements 42b of the second array segment 44b are likewise spaced apart from each other, as shown. Notwithstanding the foregoing enumeration of the eight electrode elements 42a/42b in each array segment 44, the specific number is presented by way of example only. Alternative embodiments may include additional or fewer electrode elements.

Referring now to FIG. 5, an alternative embodiment of the temporary nerve blocking device 10 incorporates a set of patch electrodes 48 that are exposed on a limb contact side 38 of the strap 12. In comparison to the first embodiment, the patch electrodes 48 are understood to correspond to the group of pin contacts 40 making up a single electrode element 42. Although the patch electrodes 48 may be constructed of any suitable material, preferably, though optionally, silver-silver chloride, or platinum-iridium may be used. The patch electrodes 48 are similarly arranged in a pattern of first and second array segments 50a, 50b. The first array segment 50a includes a first patch electrode 48a-1, a second patch electrode 48a-2, a third patch electrode 48a-3, and a fourth patch electrode 48a-4. The second array segment 50b likewise includes a first patch electrode 48b-1, a second patch electrode 48b-2, a third patch electrode 48b-3, and a fourth patch electrode 48b-4. Each of the patch electrodes 48a in the first array segment 50a are spaced apart from each other, as are the patch electrodes 48b in the second array segment 50b. As with the first embodiment of the temporary nerve blocking device 10, the specific number and arrangement of the electrode elements in the second embodiment of the temporary nerve blocking device 10 are presented by way of example, and not limited to the 4×2 matrix illustrated.

Additionally, laterally adjacent pairs of the patch electrodes similarly define electrode channels 52. For instance, the first patch electrode 48a-1 and the first patch electrode 48b-1 define a first electrode channel 52-1. Along these lines, the second patch electrode 48a-2 and the second patch electrode 48b-2 define a second electrode channel 52-2, the third patch electrode 48a-3 and the third patch electrode 48b-3 define a third electrode channel 52-3, and the fourth patch electrode 48a-4 and the fourth patch electrode 48b-4 define a fourth electrode channel 52-4.

When utilizing the second embodiment of the temporary nerve blocking device 10 an appropriate conductive gel may be applied to the surfaces of the electrode element 48 before attachment to the limb. This is understood to maintain an appropriate electrode-to-skin connection and impedance.

According to various embodiments of the present disclosure, the temporary nerve blocking device 10 is attached to the limb of the animal at specific locations, with appropriate tightness, to test for pain from a specific joint. It will be appreciated that additional cushioning or soft goods may be needed for proper placement and retention, as it is expressly contemplated that the temporary nerve blocking device 10 remains on the animal while observing movement.

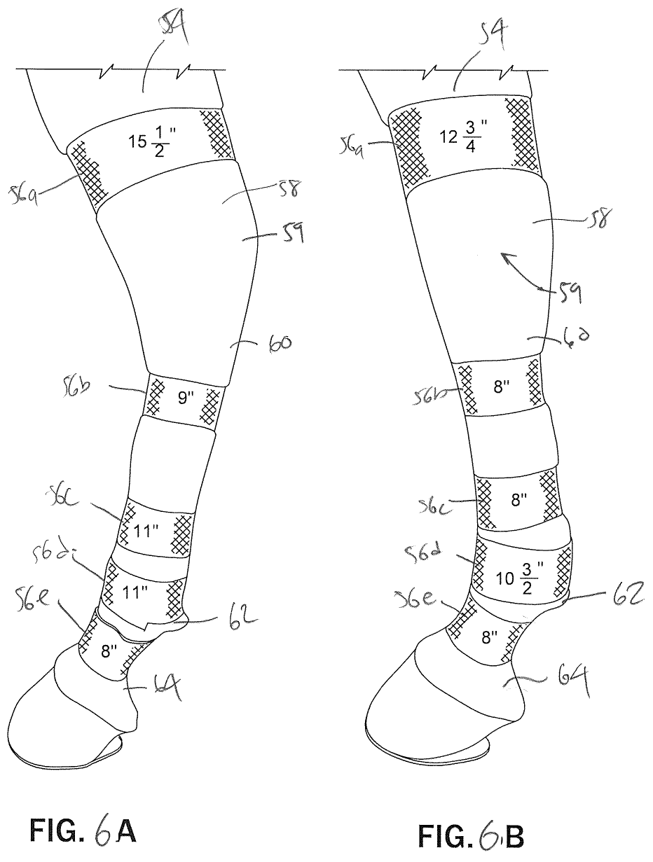

Referring to FIGS. 6A and 6B, the temporary nerve blocking device 10 may be positioned around the limb 54, which in the illustrated example is a hind at various locations thereof. There may be a first location 56a at a point along the tibia bone 58 above the hock 59, as well as a second location 56b below the hock 59 along the cannon 60. Below that may be a third location 56c on the lower end of the cannon 60, followed by a fourth location 56d coinciding with the fetlock/fetlock joint 62. There may also be a fifth location 56e below the fetlock joint 62 and coinciding with the pastern 64.

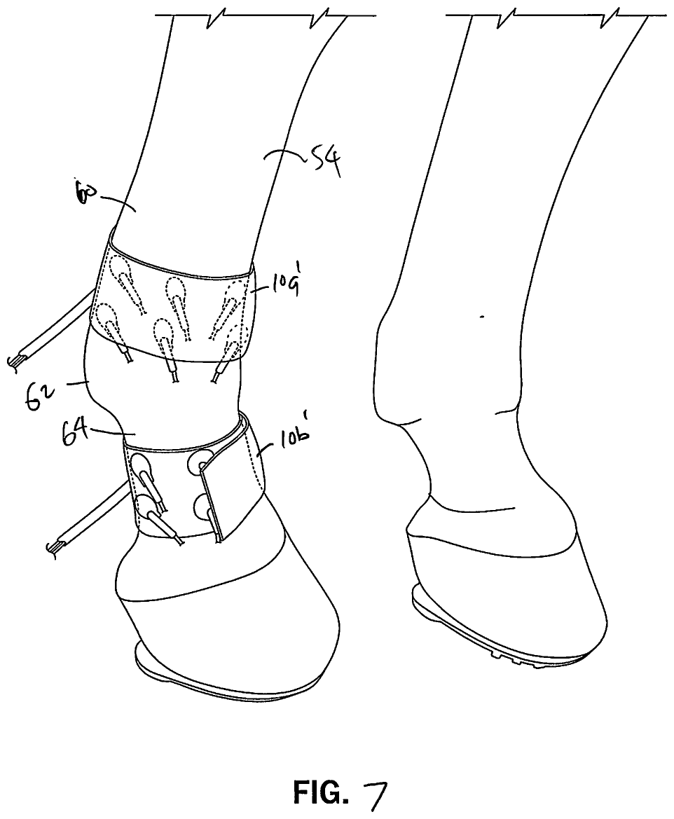

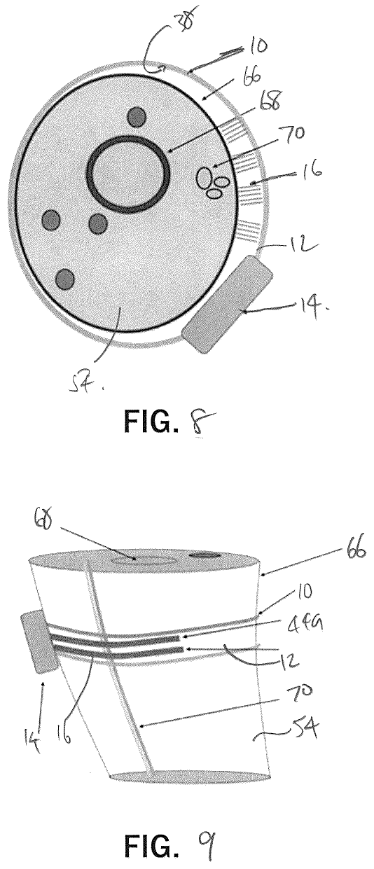

FIG. 7 illustrates the second embodiment of the temporary nerve blocking device 10′ attached to right hindleg/limb 54 of the horse. Specifically, there is a first temporary nerve blocking device 10a attached above the fetlock joint 62 on the cannon, and a second temporary nerve blocking device 10b attached below the fetlock joint 62 on the pastern 64.

FIGS. 8 and 9 illustrate additional details of the temporary nerve blocking device 10 attached to the limb 54, with the strap 12 wrapping around the limb 54 against the skin 66. The limb contact side 38 of the strap 12 abuts against the skin 66, as do the electrodes 16. Specifically illustrated in FIG. 9 is the first array segment 44a, as well as the second array segment 44b. Inside the limb 54 is a bone segment 68, as well as one or more nerves 70 that extend longitudinally along the limb 54. The nerves 70 may be positioned on the periphery of the limb 54 and away from the bone segment 68.

According to various embodiments of the present disclosure, an electrical signal, referred to herein as a nerve blocking signal is passed to the electrodes 16, which then applies the same to those parts of the limb 54 contacting the electrodes 16. This is contemplated to stop the nerves 70 from further transmitting pain signals to the brain that originate beyond the point at which the nerve blocking signal is applied.

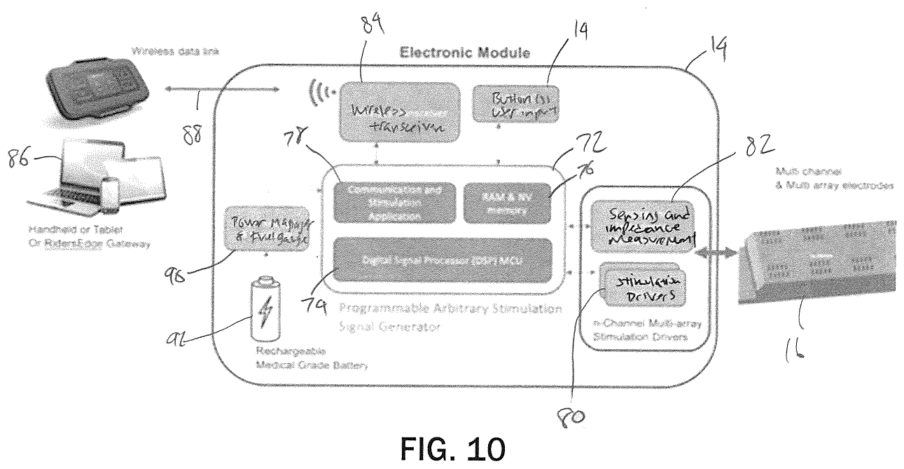

Again, the nerve blocking signal is generated by the controller 14, the details of which will now be considered. Referring to the block diagram of FIG. 10, the nerve blocking signal applied to the limb 54 through the electrodes 16 of the temporary nerve blocking device 10 may be characterized by a number of different parameters, including frequency, amplitude, phase, duration, and envelope. One or more of these parameters may be varied to affect temporary blocking of the nerves 70 from transmitting neural signals. The change or adjustment to these parameters may be based upon or are a function of one or more parameters associated with the limb 54 itself.

The nerve blocking signal originates from a programmable signal generator 72, which may further include a digital signal processor (DSP) microcontroller unit (MCU) 74. Also referred to as the DSP integrated circuit, the MCU 74 has one or more inputs and one or more outputs, and can execute pre-programmed instructions that may change its execution sequence based upon the inputs. In order to store such instructions, as well as the specifics of the nerve blocking signal that is to be generated, the programmable signal generator 72 includes a memory 76, which may include both volatile Random Access Memory (RAM) as well as non-volatile (NV) memory to more permanently store instructions and data. The pre-programmed instructions may be provided as a communications and stimulation application 78.

The nerve blocking signals that are output from the MCU 74 are understood to have low voltage and current, and so the temporary nerve blocking device 10 includes driver circuits 80 that amplify the low power nerve blocking signal before transmission to the electrodes 16. In some embodiments, each of the electrode elements 42 is understood to be controlled independently, so in such implementations, there is one driver circuit 80 for one electrode element. In some cases, the entire array segment 44 is controlled simultaneously (that is, the same signal is output on multiple electrode elements 42). This simultaneous control may be on a channel-by-channel basis, where individual pairs of the laterally adjacent electrode elements 42 defining the electrode channel 46 are simultaneously controlled. In such case, each such electrode channel 46 may have a dedicated driver circuit 80.

It is understood that nerve functions and conduction properties thereof may be temperature-dependent. That is, lower temperature may be correlated to slower and more attenuated nerve conduction. Accordingly, embodiments of the temporary nerve blocking device 10 may have functionality for adjusting the temperature of the nerve for the purpose of nerve blocking. For example, the Peltier effect, or the cooling of one junction and the heating of another may be realized by passing a signal/current through two dissimilar conductors or semiconductors. Relying upon such a conductor or semiconductor that is selectively activated by the controller 14, the temperature of the nerve 70 may be lowered, either at the situs of the electrode 16 making contact with the limb 54 (and more specifically, the nerve 70 thereof).

In addition to electrical stimulation, the temporary nerve blocking device 10 may be adapted to apply electromagnetic fields to the limb 54. Such electromagnetic fields are understood to affect the motion of charged particles, particularly those associated with neural conduction like sodium and potassium ions. Specific electromagnetic fields may therefore interact with nerve signal conduction, and can shape stimulation properties. Accordingly, the temporary nerve blocking device 10 may incorporate electromagnets that are similarly affixed to the strap 12 and connected to the controller 14 to receive stimulating signals therefrom.

Thus far, only the use of the electrodes 16 as a signal output modality has been described. It is also understood that the electrodes 16 can serve as sensor contacts/input modalities, and the sensor data captured from such source can be used to modify the nerve blocking signal. In particular, the electrodes 16 can also measure skin conductivity, and the programmable signal generator 72 can be configured to assess optimal skin impedance. To this end, the temporary nerve blocking device 10 may incorporate a sensor monitor 82 that makes skin impedance readings from the electrodes 16 connected thereto. The sensor monitor 82 may output impedance values, or at least values representative of the skin impedance as measured by the electrodes 16, to the programmable signal generator 72.

Proper nerve stimulation may be dependent on the placement of the electrodes 16 and maintaining such skin impedance, so the readings are regularly evaluated by the programmable signal generator 72 to ensure that they remain within acceptable thresholds. When the measured skin impedance exceeds the threshold, an alert can be generated. Beyond this evaluation, and with the skin impedance being maintained below the maximum threshold, the electrodes 16 can also be used to sense for neural activities in specific electrode channels 52.

Upon identifying the optimal combination of the electrodes 16 that are closest to the nerve bundle of interest, the nerve blocking signal, also referred to as a blocking stimulation, can be triggered as desired. More generally, specific electrodes 16 may be activated with the nerve blocking signal in coordination with the skin conductivity measurements made by the sensor monitor 82. One embodiment of the temporary nerve blocking device 10 contemplates a remote activation of the nerve blocking signal. Furthermore, in such embodiment, it is also possible for the parameters of such nerve blocking signal, including the aforementioned frequency, amplitude, phase, duration, and envelope parameters, to be set remotely, that is, apart from the animal without being tethered thereto. Accordingly, the temporary nerve blocking device 10 may incorporate a wireless transceiver module 84 that communicates with a remote computer system 86. The wireless transceiver module 84 may implement either or both Bluetooth® or WiFi wireless communications modalities to establish a data communications link 88 to the controller 14, and is controlled by the programmable signal generator 72. From the remote computer system 86, instructions as well as signal parameter data may be transmitted to the controller 14. The operational status, as well as feedback on the parameters of the nerve blocking signal may be transmitted back to the remote computer system 86.

While most of the high-level control over the operation of the temporary nerve blocking device 10 is contemplated to be initiated through the remote computer system 86, as indicated above, a few basic inputs/controls are onboard the controller 14. The button 26, for instance, may include a power-on/power-off function, an input to begin pairing the Bluetooth node or join a WiFi network, and the like. These input devices are connected to the programmable signal generator 72, with the signals generated thereby changing the execution of the stimulation application 78 to initiate such functions.

Either an application being executed on the remote computer system 86, or the communication and stimulation application 78 may compute the optimal stimulation voltage needed for the particular nerve blocking function that has been initiated by the veterinary practitioner. The signal parameters may be derived as a function of the measured skin impedance, the estimated nerve depth, and other dynamic as well as predefined parameters, all broadly referred to as limb parameters. Those skilled in the art will recognize other parameters that may warrant a modification to the specifics of the nerve blocking signal.

As control over the operation of individual electrode elements 42, array segments 44, and electrode channels 46 is granular, specific elements, segments, or channels may be selectively activated to achieve optimal nerve blocking. Moreover, the aforementioned signal characteristics such as frequency, waveform type (square, sawtooth, sinusoidal, etc.), amplitude, duration and envelope parameters can be selected to optimize treatment performance.

In some embodiments, the nerve blocking signal may include single as well as multiple frequencies simultaneously at various amplitudes and phases. Wide-band spectrum of signals such as band-shaped white noise and other signals may be generated and output to the electrodes 16 to achieve broad hyper ion-channel stimulation and ion channel blocking on the nerve synapse. Overall, the nerve blocking signal is contemplated to temporarily achieve sensory nerve conduction blocking. During operation, the stimulation current, including its peak and average values, are monitored and maintained during the nerve blocking function.

According to another aspect of the present disclosure, a nerve blocking efficacy test may be performed. The controller 14 is transitioned to a sensing mode that detects the signal levels from the nerve between the first array segment 50a and the second array segment 50b. A successful blocking operation is understood to result in a difference between the voltage measured by the first array segment 50a and the second array segment 50b.

As the various components in the controller 14 are electronic, it also incorporates a power management and delivery circuit 90. The controller 14 incorporates a power source 92, e.g., a battery, which may be of a medical grade. The power management and delivery circuit 90 conditions and regulates the power signal from the power source to each of the components, including the programmable signal generator 72 and its constituent elements, the driver circuit 80, and the sensor monitor 82.

The embodiments of the temporary nerve blocking device 10, are applied to an animal and preferably an equine distal extremity, for the purpose of lameness detection without the need for nerve numbing agents.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A temporary nerve blocking device attachable to a limb, the device comprising:
a strap for wrapping around the limb and defined by a limb contact side and an external side;
an array of electrodes fixed to the strap with electrical contacts thereof being exposed on the limb contact side of the strap, the electrodes being spaced apart along the strap and arranged in a predetermined pattern;
a sensor monitor coupled to the strap and connected to one or more of the electrodes, skin conductivity being measured by the sensor monitor; and
a signal generator coupled to the strap having a plurality of electrode outputs connected to a respective one of the electrodes of the array, the signal generator outputting a nerve blocking signal to one or more of the electrodes, parameters of the nerve blocking signal being a function one or more limb parameters.

2. The temporary nerve blocking device of claim 1, wherein specific ones of the electrodes are activated with the nerve blocking signal in coordination with the skin conductivity measurements therefrom.

3. The temporary nerve blocking device of claim 1, wherein one of the one or more limb parameters is measured skin impedance.

4. The temporary nerve blocking device of claim 1, wherein one of the one or more limb parameters is estimated nerve depth.

5. The temporary nerve blocking device of claim 1, where the array of electrodes is defined by a first array segment with electrodes thereof being arranged along a first side of the strap, and a second array segment with electrodes thereof being arranged along a second side of the strap opposite the first side.

6. The temporary nerve blocking device of claim 5, wherein given pairs of laterally adjacent electrodes in the first array segment and the second array segment define electrode channels.

7. The temporary nerve blocking device of claim 6, wherein the nerve blocking signal is selectively output to electrodes of different electrode channels.

8. The temporary nerve blocking device of claim 5, wherein the nerve blocking system is selectively output to different electrodes of the first array segment and the second array segment.

9. The temporary nerve blocking device of claim 1, wherein the electrodes sense nerve signals detected from the limb upon deactivation of the signal generator from outputting the nerve blocking signal, the nerve signal being transmitted to the sensor monitor for an evaluation of nerve blocking efficacy.

10. The temporary nerve blocking device of claim 1, wherein the signal generator includes:
a digital signal processor programmable to generate multiple variants of the nerve blocking signal, each variant of the nerve blocking signal being defined by one or more of the parameters of waveform type, frequency, amplitude, and phase, and duration; and
a memory for storing parameter data for the multiple variants of the nerve blocking signal.

11. The temporary nerve blocking device of claim 10, further comprising an onboard power supply powering the digital signal processor.

12. The temporary nerve blocking device of claim 1, further comprising driver circuits connected to the electrode outputs of the signal generator and amplifying the nerve blocking signal for transmission to the electrodes.

13. The temporary nerve blocking device of claim 1, further comprising:
a wireless transceiver in communication with a remote data processing device, instructions to start and stop generating the nerve blocking signal being received through the wireless transceiver from the remote data processing device.

14. The temporary nerve blocking device of claim 1, further comprising an array of electromagnets fixed to the strap and arranged in a predetermined pattern, the signal generator including a plurality of electromagnet outputs connected to a respective one of the electromagnets of the array.

15. The temporary nerve blocking device of claim 1, wherein the electrodes are pin electrodes, with each of the electrodes include a plurality of pins.

16. The temporary nerve blocking device of claim 1, wherein the electrodes are patch electrodes.

17. A temporary nerve blocking device comprising:
a strap for wrapping around a limb;
a plurality of electrodes fixed to the strap with electrical contacts thereof being exposed;
a digital signal processor integrated circuit with one or more inputs and one or more outputs, the digital signal processor being programmable to generate to the one or more outputs multiple variants of a nerve blocking signal, each variant of the nerve blocking signal being defined by one or more of the parameters of waveform type, frequency, amplitude, and phase, and duration;
a memory for storing parameter data for the multiple variants of the nerve blocking signal;
a sensor monitor connected to the electrodes and to one of the one or more inputs; and
driver circuits connected to the outputs of the digital signal processor and amplifying the nerve blocking signal for transmission to the electrodes.

18. The temporary nerve blocking device of claim 17, further comprising an onboard power supply powering the digital signal processor integrated circuit, the memory, the sensor monitor, and the driver circuits.

19. The temporary nerve blocking device of claim 17, further comprising:
a wireless transceiver in communication with a remote data processing device, instructions to start and stop generating the nerve blocking signal being received through the wireless transceiver from the remote data processing device and communicated to the digital signal processor integrated circuit through one of the one or more inputs thereof.

20. The temporary nerve blocking device of claim 17, further comprising an onboard input device connected to another one of the one or more inputs of the digital signal processor.
